(19) United States
(12) Patent Application Publication
(10) Pub. No.: US 2009/0087408 A1
(43) Pub. Date: Apr. 2, 2009

(54) Stable, non-aqueous, single-phase gels and formulations thereof for delivery from an implantable device

(75) Inventors: Stephen Berry, Hollister, CA (US); Pamela Fereira, Santa Clara, CA (US); Gunjan Junnarkar, Palo Alto, CA (US); Michael Desjardin, Sunnyvale, CA (US)

Correspondence Address: Intarcia Therapeutics, Inc.;ATTN: Barbara G. McClung, 24650 Industrial Blvd, Hayward, CA, 94545, US

(73) Assignee: INTARCIA THERAPEUTICS, INC.

(21) Appl. No.: 12/315,194

(22) Filed: Nov. 26, 2008

Related U.S. Application Data
(63) Continuation of application No. 10/742,730, filed on Dec. 19, 2003, now abandoned.
(60) Provisional application No. 60/435,180, filed on Dec. 19, 2002.

(51) Int. Cl. A61K 38/00; A61K 38/21

(52) U.S. Cl. 424/85.4; 514/2

(57) ABSTRACT

The present invention provides a suspension vehicle and suspension formulations deliverable from an implantable delivery device. In particular, the suspension vehicle of the present invention allows the formulation of beneficial agent suspensions that are stable over time at ambient and physiological temperatures. In addition, the beneficial agent suspensions formed using the suspension vehicle of the present invention allow controlled delivery of beneficial agent from an implanted delivery device over sustained periods of time, even when such delivery occurs at low flow rates, through a small-diameter delivery channel. Also included in the present invention are implantable delivery devices.

This Application claims the benefit of U.S. Provisional Application No. 60/435,180, filed Dec. 19, 2002.

### FIELD OF THE INVENTION

The present invention relates to non-aqueous, single-phase suspension vehicles that are biodegradable or biocompatible, exhibit viscous fluid characteristics suitable for suspending beneficial agents, and provide substantially uniform dispensing of beneficial agent from an implantable device. In particular, the present invention provides non-aqueous, single-phase suspension vehicles that are substantially formed using non-polymeric material, the suspension vehicles of the present invention being suitable for formulating beneficial agent suspensions that are stable over time and allow substantially uniform dispensing of beneficial agent from an implantable device at a controlled rate.

### STATE OF THE ART

Implantable devices that provide controlled delivery of beneficial agents over prolonged periods of time are known in the art. Exemplary implantable devices are taught in U.S. Pat. Nos. 5,034,229, 5,057,318, 5,110,596, and 5,782,396, the contents of which are incorporated herein by reference. Other exemplary implantable devices regulator-type implantable pumps that provide constant flow, adjustable flow, or programmable flow of beneficial agent formulations, which are available from, for example, Codman of Raynham, Mass., Medtronic of Minneapolis, Minn., and Tricumed Medinzintechnik GmbH of Germany. Further examples of implantable devices are described in U.S. Pat. Nos. 6,283,949, 5,976,109, 5,836,935, 5,511,355, which are incorporated herein by reference. Controlled delivery of a beneficial agent from an implantable device over prolonged periods of time has several potential advantages. For instance, use of implantable delivery devices generally assures patient compliance, as implantable devices are not easily tampered with by the patient and can be designed to provide therapeutic doses of beneficial agent over periods of weeks, months, or even years without patient input. Moreover, because an implantable device may be placed only once during its functional life, implantable devices may offer reduced site irritation, fewer occupational hazards for patients and practitioners, reduced waste disposal hazards, decreased costs, and increased efficacy when compared to other parenteral administration techniques, such as injections, that require multiple administrations over relatively short time intervals. However, providing controlled delivery of beneficial agents from implantable devices presents several technical challenges, and controlled delivery of peptides, polypeptides, proteins and other proteinaceous substances, such as viruses and antibodies (collectively referred to herein as “proteins”), over sustained periods of time from implantable devices has proven particularly difficult.

In order to deliver a beneficial agent from an implanted device at a controlled rate over a prolonged period of time (i.e., a period of weeks, months, or years), the beneficial agent must be formulated such that it is stable at ambient and physiological temperatures. Proteins are naturally active in aqueous environments, and preferred protein formulations have generally been aqueous solutions. However, proteins are typically only marginally stable in aqueous formulations for long durations of time, and aqueous pharmaceutical preparations of proteins have often required refrigeration or exhibited short shelf-lives at ambient or physiological temperatures. Proteins can degrade via a number of mechanisms, including deamidation, oxidation, hydrolysis, disulfide interchange, and racemization. Further, water acts as a plasticizer, which facilitates unfolding of protein molecules and irreversible molecular aggregation. Therefore, in order to provide protein formulation that is stable over time at ambient or physiological temperatures, a non-aqueous or substantially non-aqueous protein formulation is generally required.

Reduction of aqueous protein formulations to dry powdered formulations is one way to increase the stability of pharmaceutical protein formulations. For example, protein formulations can be dried using various techniques, including freeze-drying, spray-drying, lyophilization, and dessication. The dry powder protein formulations achieved by such techniques exhibit significantly increased stability over time at ambient or even physiological temperatures. However, where a flowable protein formulation is required, such as in an implantable delivery device, dry powder protein formulations alone are of limited use.

In order to provide stable, flowable protein formulations, some have suggested using solution formulations of peptides in non-aqueous, polar, aprotic solvents such as DMSO and DMF. Such formulations have shown to be stable at elevated temperatures for long periods of time. However, solvent based formulations are not useable for all protein because many proteins have low solubility in solvents that are suitable for parenteral administration, such as DMSO and DMF. As the solubility of protein in the solvent decreases, the amount of formulation required to deliver a given protein dose will increase, and though relatively large volumes of low concentration solutions of protein may be useful for delivery by injection, due to size constraints, implantable delivery devices generally require relatively high concentration protein formulations capable of delivering therapeutic levels of protein at low flow rates over prolonged periods of time.

In order to achieve a stable protein formulation of suitable protein concentration, a suspension formulation may be used. For example protein suspensions have been formulated using non-aqueous, anhydrous, aprotic, hydrophobic, non-polar vehicles, non-aqueous, protic vehicles, anhydrous pseudoplastic and thixotropic oleaginous vehicles, liposomal vehicles, and cationic lipid vehicles. Suspension formulations including particles of a protein beneficial agent dispersed within a suitable vehicle may be stable at ambient or even physiologic temperatures over prolonged periods of time, and such suspensions formulations may be prepared with relatively high concentrations of beneficial agent. However, in order for a suspension formulation to be suited to delivery of a beneficial agent at a controlled rate over sustained periods of time from an implantable device, such a suspension formulation must provide desirable stability and beneficial agent loading characterisitics. In particular, a suspension formulation suitable for use in a implantable device designed to provide controlled release of a beneficial agent over a prolonged period should also utilize a vehicle acceptable for parenteral use, maintain the beneficial agent in a substantially uniform dispersion over time, allow delivery of the suspension formulation from the implantable device, and provide ready release of the beneficial agent from the suspension formulation upon delivery to an environment of administration.

Maintaining a substantially uniform dispersion of beneficial agent over time facilitates controlled delivery of the beneficial agent from an implanted device and may work to increase stability of the beneficial agent dispersed within the suspension. If the beneficial agent dispersed within a suspension loaded into an implantable device settles over time, the concentration of beneficial agent within the suspension becomes non-uniform and the amount of beneficial agent delivered from the implantable device during its functional life may vary significantly. Such variances may cause the amount of beneficial agent delivered from an implanted device to exceed recommended dosing regimens or, alternatively, cause the amount of beneficial agent delivered to fall below therapeutic levels. Moreover, as particles of beneficial agent settle out of suspension, their association one with another increases, which can significantly increase the potential for degradation of the beneficial agent. Therefore, a suspension formulation that maintains a substantially uniform dispersion of beneficial agent over the life of the implantable device functions to both facilitate uniform delivery of the beneficial agent over time and to maintain the stability of the beneficial agent within the suspension.

In order to maintain a substantially uniform dispersion of beneficial agent in a suspension formulation, it has been found that the vehicle used to formulate the suspension should exhibit a relatively high viscosity. Depending on the particle size of the beneficial agent, a vehicle having a viscosity of about 1,000 poise or more at physiologic temperature may be required to prevent settling of the beneficial agent dispersed within a suspension formulation. It has been reported that polymer materials, such as polyvinylpyrrolidone, may be used to provide suspension vehicles that not only allow the formulation of relatively high concentration protein suspensions that are stable over time, but also offer the viscosity required to maintain a substantially uniform dispersion of protein particles. To achieve high viscosity vehicles using polymer materials, the polymer may be dissolved in a non-aqueous solvent to create single phase, viscous solution. There are few viscosity enhancing polymers that are biocompatible, and of the viscosity enhancing polymers that are biocompatible not all are sufficiently soluble in non-aqueous solvent to provide a suspension vehicle of desired viscosity.

It has been found that where certain solvents are included in polymer suspension vehicles used to form protein suspensions for delivery from an implantable device through a small delivery channel, the polymer contained in the protein suspension may precipitate within the delivery channel, causing a blockage. Where this occurs, it is believed that polymer contained within the beneficial agent suspension migrates into the aqueous environmental fluid at the interface between the aqueous environmental fluid and the protein suspension. The migration of polymer material from the protein suspension and into the aqueous environmental fluid causes a change in the composition of the protein suspension, and as the polymer dissolves into the aqueous environmental fluid within the confines of the delivery channel, a high aqueous concentration of polymer is localized within the delivery channel, causing the polymer to precipitate and potentially form a blockage. In addition, it has been found in some instances that suspensions formed using polymeric suspension vehicles may allow the ingress of aqueous fluid through the delivery channel provided in an implantable device and into the reservoir containing the protein suspension.

An alternative approach to formulating a protein suspension deliverable from an implantable device is to use a suspension vehicle formed of a blend of similar materials with a mixture of molecular weights, instead of a single-phase polymer system. Mixtures of materials such as polyethylene glycol (PEG), hydrogenated vegetable oils, and Pluronics can be used to achieve highly viscous suspension vehicles. However, as pressures sufficient to drive highly viscous materials from a delivery device are applied to multiphase suspension vehicles, separation of the relatively lower and relatively higher molecular weight fraction of the suspension vehicles may occur. As the fractions separate under the applied pressure, the lower molecular weight fractions are delivered first from the implanted device, while the higher molecular weight fractions and the beneficial agent suspended therein are left behind in the delivery device. Thus, it would be advantageous to provide a substantially non-polymeric, single-phase suspension vehicle that provides the stability and delivery characteristics necessary to deliver beneficial agents, such as peptides and proteins, from an implantable delivery device at a controlled rate over a prolonged period of time.

### SUMMARY OF THE INVENTION

The present invention provides a suspension vehicle and suspension formulations deliverable from an implantable delivery device. In particular, the suspension vehicle of the present invention allows the formulation of beneficial agent suspensions that are stable over time at ambient and physiological temperatures. In addition, the beneficial agent suspensions formed using the suspension vehicle of the present invention allow controlled delivery of beneficial agent from an implanted delivery device over sustained periods of time, even when such delivery occurs at low flow rates, through a small-diameter delivery channel.

The present invention also includes implantable delivery devices. An implantable delivery device according to the present invention may be any implantable device capable of delivering a suspension formulation of the present invention at a controlled rate over a prolonged period of time after implantation in a subject. In one aspect, the implantable delivery device of the present invention includes an osmotically driven implantable device. In another aspect, the implantable delivery device of the present invention includes a regulator-type implantable pump that provides constant flow, adjustable flow, of programmable flow of a suspension formulation of the present invention.

### BRIEF DESCRIPTION OF THE DRAWINGS

FIG. 1 illustrates an exemplary substituted sucrose ester, SAIB, which can be used to provide a suspension vehicle according to the present invention.

FIG. 2 provides a graph illustrating the release of omega-interferon from osmotic pumps delivering a beneficial agent suspension according to the present invention.

FIG. 3 provides a graph illustrating the release of omega interferon from osmotic pumps delivering a second beneficial agent suspension according to the present invention.

Table 1 provides various physical properties of SAIB.

Table 2 provides data regarding the stability of omega-interferon included in a first beneficial agent suspension according to the present invention.

Table 3 provides data regarding the stability of omega-interferon included in a second beneficial agent suspension according to the present invention.

### DETAILED DESCRIPTION OF THE INVENTION

The present invention includes non-aqueous suspension vehicles. Suspension vehicles of the present invention are single-phase, viscous, and flowable compositions that are substantially formed of hydrophobic, non-polymeric materials. As it is used herein, the term “substantially formed” indicates that the suspension vehicle is about 75 wt % to about 100 wt % hydrophobic, non-polymeric material, and the term “single-phase” indicates a homogeneous system, that exists as a distinct and mechanically separate portion in a heterogeneous system and that is both physically and chemically uniform throughout under both static and dynamic conditions.

By substantially forming the suspension vehicles of the present invention using a non-polymeric material, a single phase suspension vehicle that exhibits reduced potential for phase separation or precipitation of vehicle components can be achieved. Non-aqueous, hydrophobic, non-polymeric materials suitable for forming suspension vehicles according to the present invention include, but are not limited to, hydrophobic saccharide materials, organogels, or lipid materials that behave as single phase vehicles. A suspension vehicle of the present invention may be formed of one or more components providing a single phase, viscous gel, as defined herein. In one embodiment, the suspension vehicle of the present invention is formed of a single hydrophobic, non-polymeric material. In another embodiment, the suspension vehicle of the present invention is a viscous gel formed using two or more non-polymeric materials, including two or more hydrophobic saccharide, organogel, or lipid materials. Exemplary saccharide materials that may be used in formulating a suspension vehicle of the present invention include, but are not limited to, substituted sucrose esters that exist as fluids at ambient or physiological temperatures, such as sucrose acetate isobutyrate (“SAIB”). The suspension vehicles of the present invention allow the formulation of beneficial agent suspensions that are stable at ambient and physiological conditions and are capable of maintaining substantially uniform dispersions of beneficial agent.

In each embodiment, the suspension vehicle of the present invention is a viscous fluid or gel-like material. As it is used herein, the term “viscous fluid” refers to a flowable fluid, gel or gel-like material having a viscosity within a range of about 500 to 1,000,000 poise as measured by a parallel plate rheometer at a shear rate of 10⁻⁴/sec and 37° C. The term “viscous gel” includes Newtonian and non-Newtonian materials. Preferred are gels with a viscosity of about 1,000 to 30,000 poise as measured by a parallel plate rheometer at a shear rate of 10⁻⁴/sec and 37° C. Viscous suspension vehicles allow the creation of beneficial agent suspensions capable delivering beneficial agent at a substantially uniform rate over prolonged periods of time as the suspension is expelled from an implantable delivery device at a controlled rate.

If desired, the suspension vehicle of the present invention may include an amount of other excipients or adjuvants, such as surfactants, antioxidants, stabilizers, and viscosity modifiers. Exemplary materials that may be included in a suspension vehicle of the present invention to achieve a desired quality or performance characteristic include ethanol, propylene glycol, and IPA. Moreover, if desired, the suspension vehicle of the present invention may even incorporate one or more polymeric materials. However, where the suspension vehicle of the present invention includes an amount of polymeric material, the amount of polymeric material is relatively small and is typically chosen to reduce or eliminate any phase separation or precipitation of the polymer out of suspension vehicle as a beneficial agent suspension formed using the vehicle comes in contact with an aqueous fluid in a delivery channel. Where a suspension vehicle of the present invention includes one or more excipients or adjuvants, the amount of excipient or adjuvant included will depend on, among other factors, the type of non-polymeric material included in the vehicle, the amount and type of beneficial agent to be included in the vehicle, the adjuvant or excipient added, and the stability or flow rate characteristics desired. Regardless of the type of adjuvant or excipient used, adjuvant and excipient materials included in the suspension vehicle of the present invention will account for no more than about 25 wt % of the suspension vehicle, and in preferred embodiments where excipients or adjuvants are used, the suspension vehicle of the present invention includes no more than about 15 wt %, 10 wt % or 5 wt % adjuvant and excipient material. Whether or not it is formulated to include one or more excipients or adjuvants, a suspension vehicle of the present invention may be formulated using standard means or methods well known in the art.

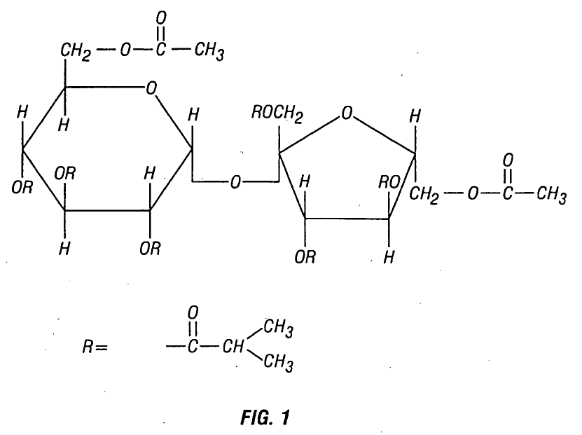

In a preferred embodiment, a suspension vehicle of the present invention is substantially formed of sucrose acetate isobutyrate (SAIB). SAIB is a hydrophobic liquid exhibiting high viscosity and limited water solubility and is commercially available. The structure of SAIB is shown in FIG. 1. SAIB has a viscosity of approximately 3,200 poise at 37° C., and is produced by the controlled esterification of sucrose with acetic and isobutyric anhydrides. SAIB metabolizes into sucrose, acetic acid and isobutyric acid. Moreover, it has been found that, when used as a suspension vehicle, SAIB provides viscous protein suspensions that are deliverable at desired rates into an aqueous environment. Suspension vehicles formed using SAIB have also been found to reduce or prevent migration of aqueous fluid from an environment of use into a reservoir of beneficial agent suspension through a delivery channel included in an implantable delivery device.

Where SAIB is used to form a suspension vehicle of the present invention, the amount of SAIB included in a suspension vehicle of the present invention may vary. If desired, the suspension vehicle may be formed entirely of SAIB. Alternatively, a single-phase suspension vehicle according to the present invention may be formed using SAIB in combination with one or more additional components. For instance, ethanol or IPA may be included in an SAIB suspension vehicle of the present invention. However, where additional components are included in an SAIB suspension vehicle of the present invention, those components account for no more than 25 wt % of the suspension vehicle, with SAIB accounting for 75 wt % or more. Preferably, an SAIB vehicle according to the present invention includes at least about 85 wt % SAIB, and even more preferably about 90 wt % or more SAIB.

In another aspect, the present invention includes a beneficial agent suspension formed using a non-polymeric suspension vehicle of the present invention. A beneficial agent suspension according to the present invention includes a beneficial agent dispersed within a suspension vehicle of the present invention. A beneficial agent suspension of the present invention may be loaded with varying amounts of beneficial agent to provide a formulation that allows dosing of the beneficial agent at a desired rate over a chosen period of time. Preferred beneficial agent suspensions according to the present invention includes about 0.1 wt % to about 15 wt % beneficial agent, depending on the potency of the beneficial agent, and more preferably, a suspension of the present invention includes from about 0.4 wt % to about 5 wt %. If the beneficial agent is dispersed within a suspension vehicle as a particulate material, the beneficial agent particles, which may contain varying amounts of beneficial agent and one or more excipients or adjuvants, preferably account for no more than about 25 wt % of the beneficial agent suspension.

A beneficial agent suspension according to the present invention is also formulated to allow dispensing from an implantable device at a desired flow rate. In particular a beneficial agent suspension of the present invention may be formulated for delivery at flow rates of up to about 5 ml/day, depending on the beneficial agent to be delivered and the implantable device used to deliver the beneficial agent suspension. Where the beneficial agent is delivered from an osmotically driven implantable device designed to provide low flow rates, the beneficial agent suspension is preferably formulated for delivery of between about 0.5 and 5 μl/day, with flow rates of about 1.5 μl/day and 1.0 μl/day being particularly preferred.

A beneficial agent suspension according to the present invention may be prepared by dispersing a desired beneficial agent within a suspension vehicle according to the present invention using any suitable means or method known in the art. The beneficial agent may be provided in any desirable form that allows dispersion of the beneficial agent within a suspension vehicle of the present invention. However, before dispersion within a suspension vehicle of the present invention, the beneficial agent is preferably provided in a stabilized dry powder form. For example, before dispersion in a suspension vehicle according to the present invention, the beneficial agent may be provided as a dry powder material achieved through a known spray drying, freeze drying, lyophilization, or supercritical fluid process. As part of providing the beneficial agent in a stabilized dry powder using, for example, a spray drying, freeze drying, lyophilization, or supercritical fluid process, the beneficial agent may be formulated with one or more adjuvants or excipients, as is known in the art, such that the dry powder beneficial agent is not a pure material but includes desired amounts of excipient or adjuvant in addition to the beneficial agent.

As it is used herein, the term “beneficial agent” refers to any chemical entity that provides a therapeutic benefit to an animal or human subject and exhibits increased stability when formulated in a non-aqueous suspension compared to an aqueous suspension or solution.

The beneficial agent included in a suspension according to the present invention is generally degradable in water but generally stable as a dry powder at ambient and physiological temperatures. Beneficial agents that may be incorporated into a suspension according to the invention include, but are not limited to, peptides, proteins, nucleotides, polymers of amino acids or nucleic acid residues, hormones, viruses, antibodies, etc. that are naturally derived, synthetically produced, or recombinantly produced. The beneficial agent included in a suspension according to the present invention may also include lipoproteins and post translationally modified forms, e.g., glycosylated proteins, as well as proteins or protein substances which have D-amino acids, modified, derivatized or non-naturally occurring amino acids in the D- or L-configuration and/or peptomimetic units as part of their structure. Specific examples of materials that may be included in as the beneficial agent in a beneficial agent suspension of the present invention include, but are not limited to, baclofen, GDNF, neurotrophic factors, conatorikin G, Ziconotide, clonidine, axokine, anitsense oligonucleotides, adrenocorticotropic hormone, angiotensin I and II, atrial natriuretic peptide, bombesin, bradykinin, calcitonin, cerebellin, dynorphin N, alpha and beta endorphin, endothelin, enkephalin, epidermal growth factor, fertirelin, follicular gonadotropin releasing peptide, galanin, glucagon, gonadorelin, gonadotropin, goserelin, growth hormone releasing peptide, histrelin, insulin, interferons, leuprolide, LHRH, motilin, nafarerlin, neurotensin, oxytocin, relaxin, somatostatin, substance P, tumor necrosis factor, triptorelin, vasopressin, growth hormone, nerve growth factor, blood clotting factors, ribozymes, and antisense oligonucleotides. Analogs, derivatives, antagonists agonists and pharmaceutically acceptable salts of each of the above mentioned agents may also be used in formulating an active agent suspension of the present invention. Preferably, the beneficial agent provided in a suspension of the present invention exhibits little or no solubility in the chosen suspension vehicle. Where, a beneficial agent exhibits some solubility in a suspension vehicle according to the present invention, a solution formulation of the beneficial agent may be formulated using the suspension vehicle, provided the solution exhibits the desired stability and deliverability characteristics.

The present invention also includes an implantable delivery device loaded with a beneficial agent suspension of the present invention. An implantable delivery device of the present invention may be embodied by any delivery system device capable of delivering a beneficial agent suspension of the present invention at a controlled rate over a sustained period of time after implantation within a subject. An implantable delivery device according to the present invention may include, for example, an implantable osmotic delivery device as described in U.S. Pat. Nos. 5,728,396, 5,985,305, 6,113,938, 6,132,420, 6,156,331, 6,375,978, 6,395,292, the contents of each of which are incorporated herein in their entirety by reference. An implantable device according to the present invention may also include a regulator-type implantable pump as is commercially available from, for example, Codman of Raynham, Mass., Medtronic of Minneapolis, Minn., and Tricumed Medinzintechnik GmbH of Germany. Specific examples of non-osmotic implantable pumps that may be included in an implantable device of the present invention include those devices described in U.S. Pat. Nos. 5,713,847, 5,368,588, 6,436,091, 6,447,522, and 6,248,112, the contents of each of which are incorporated herein in their entirety by reference.

The present invention is further described and illustrated by way of the EXAMPLES that follow.

### EXAMPLE 1

Two suspension formulations according to the present invention were prepared using SAIB as a vehicle. Solid particles of omega-interferon were dispersed within the SAIB to form a suspension formulation. The omega-interferon particles were composed of omega-interferon, sucrose, methionine and citrate, with the ratio of omega-interferon to sucrose to methionine to citrate contained in the particles being 1:2:1:1.7 (omega-interferon: sucrose: methionine: citrate). Suspension A (also referred to as the “full dose” suspension) exhibited a particle loading of approximately 10%, which is equivalent to drug loading of 1.66%. Suspension B (also referred to as the “fractional dose” suspension) exhibited a particle loading of approximately 4%, which is equivalent to a drug loading of about 0.66%.

The suspensions were mixed in a dry box under nitrogen. For each suspension, an appropriate quantity of SAIB was weighed into a beaker. The appropriate quantity of omega-interferon particles was then weighed and added to the beaker. A hot plate was warmed to maintain a target surface temperature of 55° C., and, using a using a stainless steel spatula, the omega-interferon particles were incorporated into the SAIB over a period of about 15 minutes, while the vehicle and particle composition was warmed on the hot plate. The mixed formulations were loaded in a glass syringe and de-aerated in a vacuum oven under a vacuum pressure of about −30 Hg. Following de-aeration, the glass syringes containing the suspensions were sealed and refrigerated (2-8° C.).

### EXAMPLE 2

Stability of both the suspensions was measured after storage at 40° C. under nitrogen. Samples were tested in triplicate at t=0, 2 weeks and 1 month (2 mg omega-interferon per sample). Analysis was performed using RP-HPLC to determine purity with respect to oxidation and deamidation and using SEC to determine purity with respect to aggregation and precipitation. The results of these stability studies are presented in Table 2 and Table 3.

### EXAMPLE 3

Four sets of osmotic pumps loaded with the suspension formulations prepared according to Example 1 were prepared and studied. Two sets of the osmotic pumps prepared included diffusion moderators through which the suspension formulation was delivered. In the first set, the diffusion moderators provided a spiral shaped delivery channel (spiral DM) through which the formulation was expelled, and in the second set, the diffusion moderators provided a straight delivery channel (straight DM) through which the formulation was expelled. The other two sets of osmotic pumps included delivery orifices formed by capillary tubes.

The pumps with diffusion moderators and one set of pumps prepared with a capillary tube were loaded with Suspension B prepared according to Example 1, and the remaining set of pumps prepared with a capillary tube was loaded with Suspension A prepared according to Example 1. The pumps with diffusion moderators were intended to give an indication of suspension performance when loaded in an osmotic pump. Pumps with dynamic capillaries were intended to serve as a visual aid for observing phase behavior at the water-suspension interface formed where the suspension formulation included in the systems interfaced with the aqueous liquid present in the environment of operation. The pumps with spiral diffusion moderators served as a control.

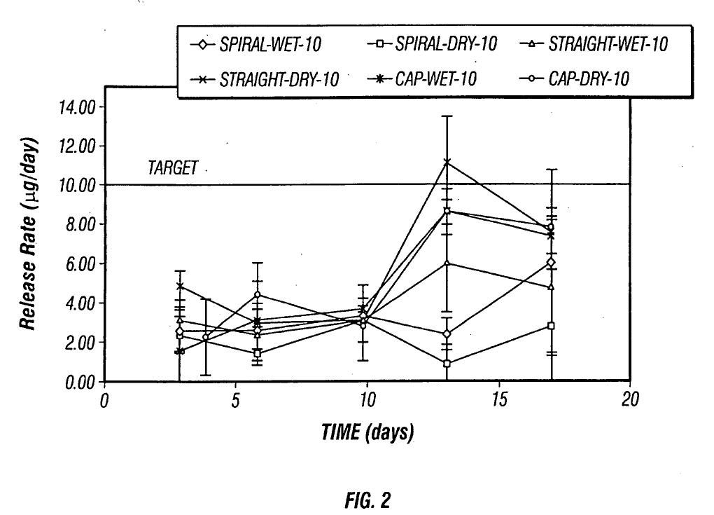

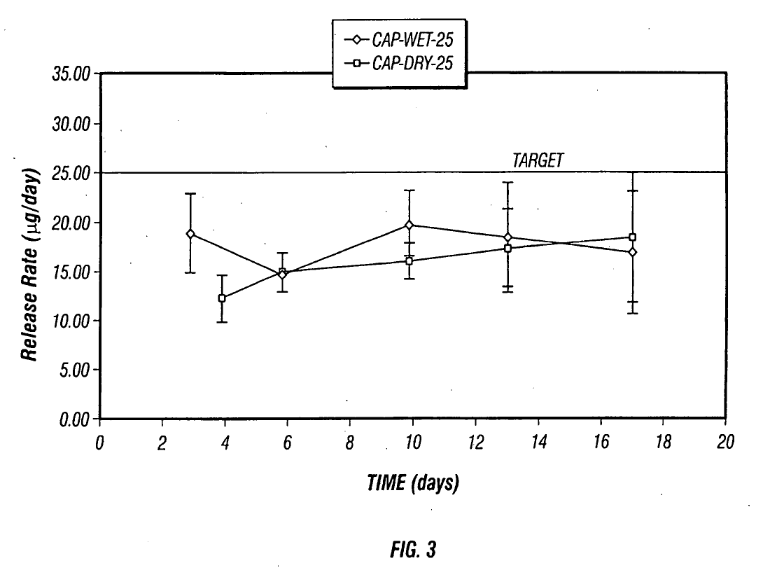

Release rate was monitored by allowing the pumps to deliver the suspension formulations into phosphate buffered saline with 0.2% sodium azide (PBS solution). Release rate performance was studied using “dry start” and “wet start” conditions. Under dry start conditions, the pumps were started and the suspension formulation was released into air until the suspension formulation emerged from the diffusion moderator or capillary tube (˜1 week), after which the diffusion moderator or capillary tube was placed into the PBS solution. Under wet start conditions, the pumps were started and the formulation release was into PBS solution (wet start) from the beginning of the study. Four pumps with a spiral DM were dry started, and four were wet started. Four pumps with a straight DM were dry started, and four were wet started. Six pumps having a capillary tube and loaded with Suspension A were dry started, and six were wet started. Six pumps having a capillary tube and loaded with Suspension B were dry started and six were wet started. The capillary tubes were observed on a weekly basis to measure the distance of PBS ingress into the formulation and observe phase changes at the interface. Omega-interferon released from the pumps (soluble and insoluble) was measured twice a week by HPLC and Advanced Protein Assay. The release rate of omega-interferon from the fractional dose suspensions is presented in FIG. 2, and the release rate of omega-interferon from the full dose suspensions is presented in FIG. 3.

TABLE 1

| | |
|---|---|
| Molecular Weight Range | 832-856 |
| Weight/Volume @ 25° C. | 1.14 kg/L |
| Flash Point, Tag Closed Cup, ° C. (° F.) | 226 (440) |
| Decomposition Temperature, ° C. (° F.) | 288 (550) |
| Solubility in Water @ 25°, wt % | 0.1 |

TABLE 2
Stability at 40° C.
Formulation 173-A (Full Dose, 10% particle loading, 1.66% drug loading)

| | RP-HPLC (n = 3) | | | |
|---|---|---|---|---|
| | Initial | | | |
| | (protein particles) | Initial | 2 weeks | 4 weeks |
| omega-IFN | 93.70 (0.31) | 90.88 (0.34) | 86.91 (0.06) | 86.32 (0.36) |
| % Oxidized | 2.99 (0.01) | 5.78 (0.05) | 8.58 (0.05) | 8.25 (0.06) |
| % Deamidated | 0.82 (0.01) | 1.15 (0.02) | 2.07 (0.02) | 2.51 (0.01) |
| % Unknown | 2.49 (0.18) | 2.19 (0.32) | 2.44 (0.02) | 2.92 (0.44) |

| | SEC (n = 3) | | | |
|---|---|---|---|---|
| | Initial | | | |
| | (protein particles) | Initial | 2 weeks | 4 weeks* |
| % Monomer | 99.92 (0.01) | 99.76 (0.03) | 99.65 (0.02) | 99.31 (0.03) |
| % Dimer | 0.08 (0.01) | 0.024 (0.03) | 0.34 (0.02) | 0.68 (0.04) |
| Unknown | ND | ND | 0.01 (0.00) | 0.01 (0.01) |

*n = 2
ND = Not detected, standard deviation in parenthesis

TABLE 3
Stability at 40° C.
Formulation 173-B (Fractional Dose, 4% particle loading, 0.66% drug loading)

| | RP-HPLC (n = 3) | | | |
|---|---|---|---|---|
| | Initial | | | |
| | (protein particles) | Initial | 2 weeks | 4 weeks |
| omega-IFN | 93.70 (0.31) | 90.74 (0.30) | 85.90 (0.37) | 84.66 (0.02) |
| % Oxidized | 2.99 (0.01) | 6.13 (0.10) | 10.51 (0.02) | 10.36 (0.02) |
| % Deamidated | 0.82 (0.01) | 0.96 (0.02) | 1.64 (0.01) | 1.96 (0.03) |
| % Unknown | 2.49 (0.18) | 2.17 (0.30) | 1.96 (0.35) | 3.03 (0.03) |

| | SEC (n = 3) | | | |
|---|---|---|---|---|
| | Initial | | | |
| | (protein particles) | Initial | 2 weeks | 4 weeks* |
| % Monomer | 99.92 (0.01) | 99.83 (0.01) | 99.67 (0.01) | 99.51 (0.41) |
| % Dimer | 0.08 (0.01) | 0.017 (0.01) | 0.32 (0.02) | 0.49 (0.41) |
| Unknown | ND | ND | 0.01 (0.01) | 0.01 (0.01) |

ND = Not detected, standard deviation in parenthesis

1-40. (canceled)

41. A pharmaceutical composition comprising:
a particle formulation comprising a protein, sucrose, methionine, and citrate, wherein the ratio of protein to sucrose to methionine to citrate is substantially 1:2:1:1.7.

42. The pharmaceutical composition of claim 41, wherein the protein is an interferon.

43. The pharmaceutical composition of claim 42, wherein the interferon is omega-interferon.

44. The pharmaceutical composition of claim 41, wherein the particle formulation is a dry powder.

45. The pharmaceutical composition of claim 44, wherein the dry powder is formed by spray drying, freeze drying, lyophilization, or supercritical fluid process.

46. The pharmaceutical composition of claim 41, further comprising a suspension vehicle.

47. The pharmaceutical composition of claim 46, wherein the particle formulation is suspended in the suspension vehicle at less than about 25 wt %.

48. The pharmaceutical composition of claim 46, wherein the particle formulation is suspended in the suspension vehicle at about 0.1 wt % to about 15 wt %.

49. The pharmaceutical composition of claim 46, wherein the particle formulation is suspended in the suspension vehicle at about 0.4 wt % to about 5 wt %.

50. An implantable osmotic delivery device, comprising the pharmaceutical composition of claim 41.

51. An implantable osmotic delivery device, comprising the pharmaceutical composition of claim 46.
